(19) United States
(12) Patent Application Publication
(10) Pub. No.: US 2010/0113610 A1
(43) Pub. Date: May 6, 2010

(54) METHODS FOR CONTROLLING INFLAMMATORY AND IMMUNOLOGICAL DISEASES

(75) Inventors: Chang H. Kim, West Lafayette, IN (US); Hyung W. Lim, West Lafayette, IN (US); Seung G. Kang, West Lafayette, IN (US)

Correspondence Address: BOSE MCKINNEY & EVANS LLP, 111 MONUMENT CIRCLE, SUITE 2700, INDIANAPOLIS, IN, 46204, US

(73) Assignee: Purdue Research Foundation, West Lafayette, IN (US)

(21) Appl. No.: 12/609,918

(22) Filed: Oct. 30, 2009

Related U.S. Application Data
(63) Continuation of application No. PCT/US2008/062125, filed on May 1, 2008.
(60) Provisional application No. 60/915,162, filed on May 1, 2007.

(51) Int. Cl. A61K 31/07; C12N 5/00; A61P 29/00; A61P 37/00

(52) U.S. Cl. 514/725; 435/375

(57) ABSTRACT

The present invention provides methods for generating mucosal tissue homing immunosuppressive T-cells comprising treating naïve T-cells with retinoids and/or retinoid agonists. Methods are also provided for treating a mammal having an inflammatory or immunological disease by administering a therapeutically effective dose of retinoids and/or retinoid agonists. Additional methods are also provided for boosting the immune system of a mammal by inhibiting the generation of immunosuppressive T-cells by administering a therapeutically effective dose of a retinoid receptor antagonist to the mammal.

### GOVERNMENT RIGHTS

This invention was made with Government support under Grant Al063064 awarded by the National Institute of Allergy and Infectious Diseases. The Government has certain rights in this invention.

### BACKGROUND OF THE INVENTION

The present invention relates generally to methods for controlling inflammatory and immunological diseases and more particularly to methods of administering retinoids for generation of immunosuppressive T cells for controlling inflammatory and immunological diseases.

Preformed vitamin A in food is absorbed in the form of retinol, which can be made into retinal and retinoic acid in the body (Chambon, 1996, Faseb J; Semba, 1994, Clin Infect Dis; Zhang and Duvic, 2003, Dermatol Ther; Mark, Ghyselinck, and Chambon, 2006, Annu Rev Pharmacol Toxicol). Also retinol can be generated from provitamin A carotenoids such as beta-carotene. Three metabolites of vitamin A, 11-cis-retinal, all-trans-retinoic acid (ATRA) and 9-cis-RA, mediate the major biological functions of the vitamin (Mark, Ghyselinck, and Chambon, 2006, Annu Rev Pharmacol Toxicol; Duester, 2000, Eur J Biochem). Vitamin A has pleiotropic functions in the body. 11-cis-retinal functions as chromophores in light absorption for vision, and retinoic acid participates in bone formation, reproduction, and differentiation of many cell types during embryogenesis [1-5]. In this regard, retinoid deficiency or over-doses can cause teratogenesis (Collins and Mao, 1999, Annu Rev Pharmacol Toxicol). Vitamin A plays important roles in fighting against pathogens. One such a function is its role in formation of epithelial linings of the body including eyes, membranes of mucosal tissues (respiratory/urinary/intestinal tracts) and skin, breakage of which leads to invasion by pathogens. Through their effects on cell differentiation and death, retinoids inhibit or reverse the carcinogenic process in some types of cancers in oral cavity, head and neck, breast, skin, liver and blood cells (Soprano, Qin, and Soprano, 2004, Annu Rev Nutr; Okuno et al., 2004, Curr Cancer Drug Targets).

All-trans-RA and 9-cis-RA function as the ligands for retinoid nuclear receptors that act as transcription factors for gene expression. As retinoid nuclear receptors, RA receptor (RAR) isotypes (α, β, and γ) and the retinoid X receptor (RXR) isotypes (α, β, and γ) have been identified (Mark, Ghyselinck, and Chambon, 2006, Annu Rev Pharmacol Toxicol). Each receptor has multiple isoforms, generated through alternative mRNA splicing and/or transcription (Chambon, 1996, Faseb J). ATRA preferentially binds RARs, whereas 9-cis-RA binds equally well to both RARs and RXRs (Mark, Ghyselinck, and Chambon, 2006, Annu Rev Pharmacol Toxicol; Szondy, Reichert, and Fesus, 1998, Cell Death Differ). The two groups of retinoid nuclear receptors form RXR/RAR heterodimers, which can function as either transcriptional repressors or activators (Bourguet, Germain, and Gronemeyer, 2000, Trends Pharmacol Sci). In the absence of RAR ligands, apo-receptors recruit corepressors such as nuclear receptor corepressor (N-CoR) or silencing mediator for retinoid and thyroid hormone receptor (SMRT), which then recruit histone deacetylases (HDACs). Binding of RAR agonists to the receptors induces active conformation of the receptors. This decreases the affinity for corepressors, and creates a binding surface for histone acetyltransferase (HAT) co-activators, such as CREB-binding protein (CBP) and p160 (e.g. TIF2). Transcriptional coactivators are involved in chromatin remodeling (decondensation due to histone acetylation) or recruitment of the basal transcription machinery. However, RXR agonists alone cannot dissociate co-repressors from heterodimers of RAR-RXR, and retinoid receptor antagonists prevent the formation of the holo-conformation (Clarke et al., 2004, Expert Rev Mol Med).

Many functions of retinoids have been identified in regulation of immune responses. As positive roles in regulation of the immune system, retinoids enhance the numbers and effector functions of neutrophils, NK cells, B cells, and Th2 cells (Stephensen, 2001, Annu Rev Nutr; Cantorna, Nashold, and Hayes, 1994, J Immunol; Racke et al., 1995, J Immunol; Hoag et al., 2002, J Nutr; Stephensen et al., 2002, J Immunol; Iwata, Eshima, and Kagechika, 2003, Int Immunol; Tokuyama and Tokuyama, 1996, Cell Immunol). Retinoids induce gut homing receptor expression in T cells (Iwata et al., 2004, Immunity) and B cell Ig switch to IgA (Tokuyama and Tokuyama, 1996, Cell Immunol), and, therefore, is an important regulator of mucosal immunity. Vitamin A is required for antibody responses to T-dependent and bacterial polysaccharide antigens (Pasatiempo et al., 1990, Faseb J; Smith and Hayes, 1987, Proc Natl Acad Sci U S A), normal IgA levels in the intestinal fluid (Sirisinha et al., 1980, Clin Exp Immunol), prevention of activation-induced T cell apoptosis (Yang, Vacchio, and Ashwell, 1993, Proc Natl Acad Sci U S A; Iwata et al., 1992, J Immunol), and normal phagocytic functions and resistance to bacterial pathogens (Iwata et al., 2004, Immunity; Wiedermann et al., 1996, Infect immun). On the other hand, retinoids decrease macrophage numbers in marrow and spleen. Vitamin A supplementation decreases inflammatory responses and tissue damage (Shams et al., 1994, Cornea). Vitamin A supplementation decreased serum concentrations of inflammatory cytokines such as TNF-α a and IL-1 but increased suppressive cytokines such as IL-10 (Szondy, Reichert, and Fesus, 1998, Cell Death Differ; Aukrust et al., 2000, Eur J Clin Invest). Consistently, supplementation with vitamin A suppressed inflammation in experimental autoimmune encephalomyelitis (Racke et al., 1995, J Immunol; Vladutiu and Cringulescu, 1968, Experientia; Massacesi et al., 1987, J Neurol Sci; Wang et al., 2000, Life Sci). Paradoxically, vitamin A deficiency is linked to both defective delayed type hypersensitivity (Sijtsma et al., 1990, Vet Immunol Immunopathol; Smith, Levy, and Hayes, 1987, J Nutr; Wiedermann et al., 1996, Scand J Immunol) and excessive Th1 cell function (Cantorna, Nashold, and Hayes, 1994, J Immunol; Racke et al., 1995, J Immunol; Hoag et al., 2002, J Nutr; Stephensen et al., 2002, J Immunol; Iwata, Eshima, and Kagechika, 2003, Int Immunol). Vitamin A is also linked to both lower and higher incidences of several types of cancers in humans (Fontham, 1990, Int J Epidemiol; Albanes et al., 1996, J Natl Cancer Inst).

Regulatory T cells were initially identified by their suppressive activity on immune response to self-antigens (Sakaguchi, 2004, Annu Rev Immunol; Baecher-Allan, Viglietta, and Hafler, 2004, Semin Immunol; Bach and Francois Bach, 2003, Nat Rev Immunol; Bluestone and Abbas, 2003, Nat Rev Immunol; Shevach et al., 2003, Novartis Found Symp), but it was found later that they can also suppress immune responses to alloantigens (Wood and Sakaguchi, 2003, Nat Rev Immunol; Albert et al., 2005, Eur J Immunol) and pathogens (Hod et al., 2002, Proc Natl Acad Sci U S A). The existence of regulatory T cells that induce immune tolerance to self antigens was first reported many decades ago (Baum, Liebermann, and Frenkel, 1969, J Immunol; Baker et al., 1970, J Immunol). Although regulatory T cells can be induced from non-regulatory T cells in the periphery, the presence of naturally generated regulatory cells is firmly established (Shevach et al., 2003, Novartis Found Symp; von Boehmer, 2005, Nat Immunol; Sakaguchi, 2005, Nat Immunol; Picca and Caton, 2005, Curr Opin Immunol). The key feature of naturally generated regulatory T cells is that they express FoxP3 (the product of the mouse FoxP3 gene) or FOXP3 (the human version)(Ziegler, 2006, Annu Rev Immunol). We will use the term “FoxP3” for both human FOXP3 and mouse FoxP3 in this application. FoxP3 is a member of the forkhead/winged-helix family of transcriptional regulators. FoxP3 confers T cells with regulatory function and increases the expression of CTLA-4 and CD25, but decreases IL-2 production by acting as a transcriptional repressor (Hori, Nomura, and Sakaguchi, 2003, Science; Kasprowicz et al., 2003, J Immunol; Fontenot, Gavin, and Rudensky, 2003, Nat Immunol; Schubert et al., 2001, J Biol Chem). FoxP3 binds to and suppresses nuclear factor of activated T cells (NFAT) and nuclear factor-kappaB (NFKB) (Bettelli, Dastrange, and Oukka, 2005, Proc Natl Acad Sci U S A). In addition to the natural FoxP3⁺ cells of thymic origin, regulatory T cells can also be generated in response to antigens in the periphery (Kretschmer et al., 2005, Nat Immunol).

Regulatory T cells play critical roles in regulation of immune response not only to self-antigens, alloantigens and tumor antigens but also to foreign antigens including pathogens in certain conditions. In animal models such as mice and rats, CD4⁺CD25⁺ T cells (commonly called Tregs; enriched with FoxP3⁺cells) have been shown to suppress a number of antigen-induced autoimmune diseases: diabetes (Salomon et al., 2000, Immunity), autoimmune encephalomyelitis (Kohm et al., 2002, J Immunol; Furtado et al., 2001, Immunol Rev), thyroiditis (Sakaguchi et al., 1995, J Immunol), inflammatory bowel disease (Singh et al., 2001, Immunol Rev) and gastritis (Sakaguchi et al., 1995, J Immunol; Suri-Payer et al., 1998, J Immunol) to name a few. They can also prevent graft rejection and graft-versus-host disease (Wood and Sakaguchi, 2003, Nat Rev Immunol; Chen et al., 2001, Transplant Proc; Oluwole et al., 2003, Transpl Immunol). Systemic lupus erythematosus (SLE) is an autoimmune disease characterized by inappropriate regulation of self-reactive B and T cells. In pediatric SLE patients, the frequency of CD4⁺CD25⁺ Regulatory T cells was significantly decreased in patients with active SLE compared with normal controls and patients with inactive SLE (Lee et al., 2006, Immunology). In collagen-induced arthritis, a popular model of rheumatoid arthritis, CD25⁺ cell depletion lead to a significantly more severe disease with higher antibody titers against collagen than that of control mice (Morgan et al., 2003, Arthritis Rheum). In infection by pathogens, regulatory T cells are involved in limiting the magnitude of immune responses to pathogens. This suppression, however, is a double edged-sword for hosts: While regulatory T cells function to limit tissue damage in the hosts, they can also help pathogens to avoid the immune system, leading to chronic infection (Rouse, Sarangi, and Suvas, 2006, Immunol Rev).

CD4⁺CD25⁺ T cells (FoxP3⁺) T cells are highly enriched in tumors, including melanoma (Ghiringhelli et al., 2004, Eur J Immunol), cervical carcinoma (Fattorossi et al., 2004, Gynecol Oncol), gastrointestinal tract cancer (Sasada et al., 2003, Cancer), gastric cancer (Kawaida et al., 2005, J Surg Res), lung cancer (Woo et al., 2001, Cancer Res), ovarian cancer (Woo et al., 2001, Cancer Res), colorectal cancer (Somasundaram et al., 2002, Cancer Res), breast cancer, pancreas adeno caricinoma (Liyanage et al., 2002, J Immunol), head and neck cancer (Schaefer et al., 2005, Br J Cancer), hepatocellular carcinoma (Unitt et al., 2005, Hepatology), and leukemia and lymphoma (adult T cell leukemia/lymphoma (Berger et al., 2005, Blood), Hodgkin's lymphoma (Marshall et al., 2004, Blood), and chronic lymphocytic leukemia (Beyer et al., 2005, Blood). A single injection of anti-CD25 (interleukin 2 receptor-α) monoclonal antibody (PC61) resulted in the regression of tumors in mice (Onizuka et al., 1999, Cancer Res). Tumor transplants in nude mice were eradicated by transferring CD25⁺ cell-depleted, but not total, spleen cells (Shimizu, Yamazaki, and Sakaguchi, 1999, J Immunol). Depletion of CD25⁺ T cells also induced anti-tumor-immunity against murine colorectal tumor CT26. Thus, FoxP3⁺ regulatory T cells represent another barrier to effective tumor immunity in animals and humans (Pure, Allison, and Schreiber, 2005, Nat Immunol).

Thus it would be desirable to have a method for controlling inflammatory and immunological diseases by generating immunosuppressive T cells where the immunosuppressive T cells target normal and inflamed mucosal tissues. It would also be desirable to provide methods of blocking immunosuppressive T cells to boost immune responses to pathogens and cancers.

### SUMMARY OF THE INVENTION

In one aspect of the present invention there is provided a method for generating mucosal tissue homing immunosuppressive T-cells comprising treating naive T-cells with a retinoid or a retinoid receptor agonist. The retinoid may be retinol palmitate, retinyl palmitate, retinol acetate, beta-carotene or combinations thereof. Alternatively, the retinoid may be all-trans-, 9-cis-, 11-cis retinoic acid or combinations thereof. The retinoid receptor agonist is AM-580, BMS641, BMS961, CD666, TTNPB, ATRA, Ro25-7386, methoprene acid or combinations thereof.

In another aspect of the present invention there is provided a method for treating a mammal having an inflammatory or immunological disease comprising administering to the mammal a therapeutically effective dose of a retinoid or a retinoid receptor agonist.

In a further aspect of the present invention there is provided a method for treating a mammal having an inflammatory or immunological disease comprising the steps of obtaining naïve T-cells from the mammal, culturing the naïve T-cells with a retinoid or a retinoid receptor agonist, generating immunosuppressive T-cells and administering the immunosuppressive T-cells to the mammal.

In yet another aspect of the present invention there is provided a method for boosting the immune response in a mammal comprising administering to the mammal a therapeutically effective dose of a retinoid receptor antagonist.

These and other features, aspects and advantages of the present invention will become better understood with reference to the following drawings, description and claims.

### BRIEF DESCRIPTION OF THE DRAWINGS

FIG. 1A shows the RT-PCR analysis of retinoic acid induced FoxP3 gene expression in CD4 T cells;

FIG. 1B is a histogram showing flow cytometry analysis of retinoic acid induced FoxP3 gene expression in CD4 T cells;

FIG. 1C is a graph showing dose-dependent induction of FoxP3 by ATRA;

FIG. 2 shows the induction of RARα by ATRA in CD4 T cells;

FIG. 3A is a histogram showing the RARα dependent induction of FoxP3⁺ cells;

FIG. 3B is a FACS dot plot showing the RARα dependent induction of FoxP3⁺ cells;

FIG. 4A is a FACS dot plot showing the cooperation of ATRA and TGF-β1 in induction of FoxP3⁺ T cells;

FIG. 4B is a graph showing RARα-independent induction of FoxP3 by TGF-β1;

FIG. 5A is bar graph showing that ATRA-treated T cells are hypo-proliferative;

FIG. 5B is a graph showing that ATRA-treated T cells are suppressive in function;

FIG. 6A is a FACS dot plot showing granzyme expression in ATRA-treated FoxP3⁺ T cells;

FIG. 6B is a bar graph showing granzyme expression in ATRA-treated FoxP3⁺ T cells;

FIG. 7 is a FACS dot plot showing that ATRA-treated FoxP3⁺ T cells efficiently kill immune cells;

FIG. 8A is a bar graph showing the expression of mucosal tissue homing receptors in vitamin A-induced FoxP3⁺ T cells;

FIG. 8B is a bar graph showing the expression of mucosal tissue homing receptors in vitamin A-induced FoxP3⁺ T cells;

FIG. 9A is a FACS dot plot showing ATRA induction of mouse FoxP3 T cells;

FIG. 9B is a FACS dot plot showing expression of homing receptors in ATRA-induced mouse FoxP3 T cells;

FIG. 9C is a FACS dot plot showing AM-580 induction of mouse FoxP3 T cells;

FIG. 10 is a bar graph showing the tissue-specific migration of retinoid-induced FoxP3⁺ T cells;

FIG. 11A is a FACS dot plot showing the regulation of mucosal FoxP3⁺ T cell numbers by vitamin A in vivo;

FIG. 11B is a FACS dot plot showing the regulation of mucosal FoxP3⁺ T cell numbers by vitamin A in vitro;

FIG. 12 is a graph showing suppression of the induction of inflammation in mice treated with retinoid-induced FoxP3⁺ regulatory T cells; and

FIG. 13 is a bar graph showing the regulation of anti-inflammatory T cell numbers in vivo by controlling vitamin A consumption in mice.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides a method for generating mucosal tissue homing immunosuppressive T-cells comprising treating naive T-cells with a retinoid or a retinoid receptor agonist. By utilizing the FoxP3 induction activity of retinoid receptor agonists, FoxP3⁺ regulatory T cells with specialized functions and migratory behavior can be generated. The selectivity of the homing capacity of retinoid-induced FoxP3⁺ regulatory T cells for mucosal tissue and their unique biological function are the advantageous for treating inflammatory and immunological diseases, particularly in mucosal tissue.

In one embodiment of the present invention mucosal tissue homing immunosuppressive T-cells may be generated by treating naïve T-cells with retinoids and/or retinoid agonists. Immunosuppressive T-cells may be generated ex vivo by isolating naïve T-cells and culturing them with retinoids or retinoid agonists. The concentration of the retinoid and/or retinoid agonist may be from about 100 pM to about 1 mM. In an exemplary embodiment, the concentration may be from about 100 pM to about 100 nM. Conventional naïve T-cells may be isolated from cord blood, peripheral blood or secondary lymphoid tissues. Non-limiting examples of secondary lymphoid tissues may be tonsils, spleen or appendix. Alternatively, immunosuppressive T-cells may be generated in vivo by administering retinoids and/or retinoid agonists to a mammal.

In another embodiment of the present invention retinoids may be Vitamin A or derivatives of Vitamin A such as, but not limited to, retinol palmitate, retinyl palmitate, beta-carotene or combinations thereof. In an exemplary embodiment, the retinoids may be all-trans-, 9-cis-, 11-cis retinoic acid or combinations thereof. In an alternate exemplary embodiment, retinoid agonists may be AM-580, BMS641, BMS961, CD666, TTNPB, ATRA, Ro25-7386, methoprene acid or combinations of these. Other agonists that may activate the receptors RAR/RXRα, β or γ are also contemplated by the present invention.

In another embodiment of the present invention, mammals having an inflammatory and/or immunological disease may be treated by administering a therapeutically effective dose of a retinoid and/or a retinoid agonist. Non-limiting examples of inflammatory or immunological diseases are Crohn's disease, ulcerative colitis, rheumatoid arthritis, multiple sclerosis, diabetes mellitus, Alzheimer's disease, Johne's disease or lupus. The mammal may be treated by directly administering the retinoid and/or retinoid agonist. In an exemplary embodiment, the retinoid and/or retinoid agonist may be administered subcutaneously, intradermally, intraperitoneally, intramuscularly, orally, intravenously, topically or a combination thereof. The medical practitioner will be able to determine the therapeutically effective dose based on the severity of the disease and patient parameters. The retinoid and/or retinoid agonist may be administered alone or may be formulated with other compounds. Non-limiting examples of compounds that may also be administered are TGF-β1, vitamin D, estrogen, progesterone or IL-10.

In one embodiment, the retinoid and/or retinoid agonist is administered directly to the mammal. In an alternate embodiment, naïve T-cells are isolated and cultured with the retinoid and/or retinoid agonist to generate immunosuppressive T-cells. The immunosuppressive T-cells are then administered to the mammal to treat the inflammatory and/or immunological diseases. The naïve T-cells may be autologous, allogenic or xenogenic.

In a further embodiment, the immune system of a mammal may be boosted by suppressing immunosuppressive T-cells. Immunosuppressive T-cells may be suppressed by administering a therapeutically effective dose of a retinoid antagonist to the mammal. A retinoid antagonist may be any compound that blocks the function of RAR and/or RXR receptors. Non-limiting examples of retinoid antagonists may be Ro41-5253, Ro13-7410, AGN193109, LG100754 or combinations of these agonists. Other antagonists that can block the receptors RAR/RXRα, β or γ are also contemplated by the present invention. Boosting a mammal's immune system may be used as a way to treat diseases caused by pathogens such as, but not limited to, viruses, bacteria or fungi. Alternatively, the method of the present invention may be used to treat cancers such as, but not limited to, cancers of the intestine, respiratory tracts, pancreas, oral cavity, nasal cavity, lungs, mammary gland or cervix.

### EXAMPLE 1

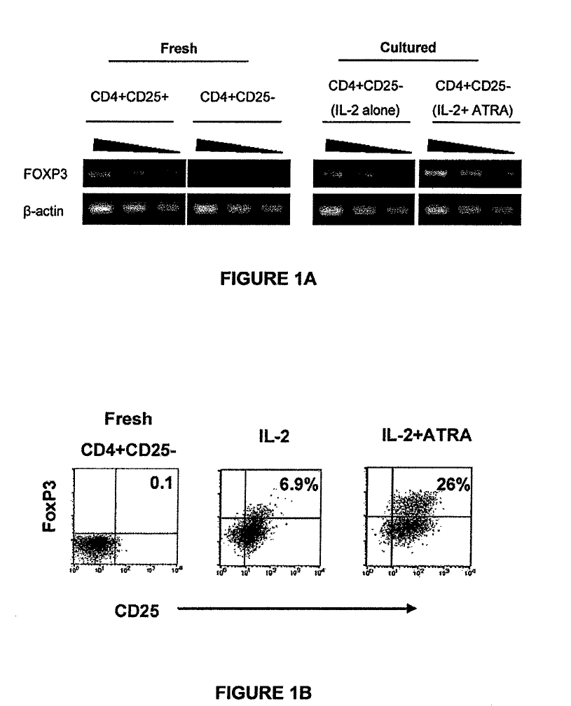

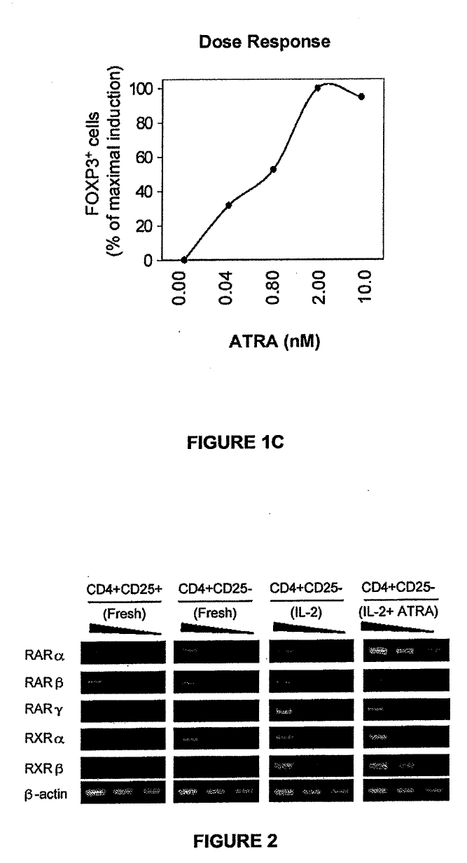

All-trans-retinoic acid (ATRA), a major vitamin A metabolite, induces FoxP3 expression in T cells. FoxP3 is the master transcription factor for CD4⁺CD25⁺ regulatory T cells. The majority of CD4⁺CD25⁺ cells are FoxP3⁺, whereas most CD4⁺CD25⁻ cells are FoxP3⁻. Neonatal human cord blood CD4⁺CD25⁻ naïve T cells were cultured in a T cell activation condition with IL-2 (25 U/ml) and phytohemagglutinin (PHA, 5 μg/m) in the presence or absence of all-trans retinoic acid (ATRA, 2 nM) for 6 days. Naïve CD4⁺CD25⁻ T cells were activated in the absence and presence of ATRA, and examined for expression of FoxP3 mRNA and protein by RT-PCR analysis (FIG. 1A), flow cytometry analysis (FIG. 1B) and dose-dependent induction of FoxP3 by ATRA (FIG. 1C). Representative data from at least three independent experiments are shown. The T cells, activated in the presence of IL-2, expressed the FoxP3 mRNA (FIG. 1A). When the T cells were cultured with both ATRA and IL-2, the levels of FoxP3 mRNA expression increased compared to IL-2 alone (FIG. 1A). Also, at the protein level, significantly more of the T cells cultured in the presence of ATRA than in its absence were FoxP3⁺ cells (FIG. 1B). These results suggest that ATRA is a novel inducer of FoxP3⁺ cells. The optimal ATRA concentration for induction of FoxP3 was 2-10 nM. The culture condition included only T cells and not other cell types, and thus, the effect of ATRA on induction of FoxP3 is thought to be direct on T cells.

### EXAMPLE 2

RARα is induced in ATRA-treated T cells. RARα, RARβ and RARγ are the major receptors that are likely to mediate the function of ATRA. The mRNA expression of RARs and RXRs in freshly isolated CD4⁺CD25⁺ and CD4⁺CD25⁻ T cells was determined, as well as in CD4⁺CD25⁻T cells activated in ATRA. Neonatal human cord blood CD4⁺CD25⁻ naïve T cells were cultured in a T cell activation condition with IL-2 (25 U/ml) and PHA (5 μg/ml) in the presence or absence of ATRA (2 nM) for 6 days. Freshly isolated CD4^{÷}CD25⁺ and CD4⁺CD25⁻ cells are shown for comparison. RT-PCR was performed, and a representative set of results out of 4 independent experiments are shown.

The most highly expressed receptor in response to ATRA was RARα among the 6 RAR and RXR family receptors. Expression of RARα & RARβ and RXR_{γ} at low levels was detected in freshly isolated CD4⁺CD25⁺ and CD4⁺CD25⁻ CD4 T cells (FIG. 2).

### EXAMPLE 3

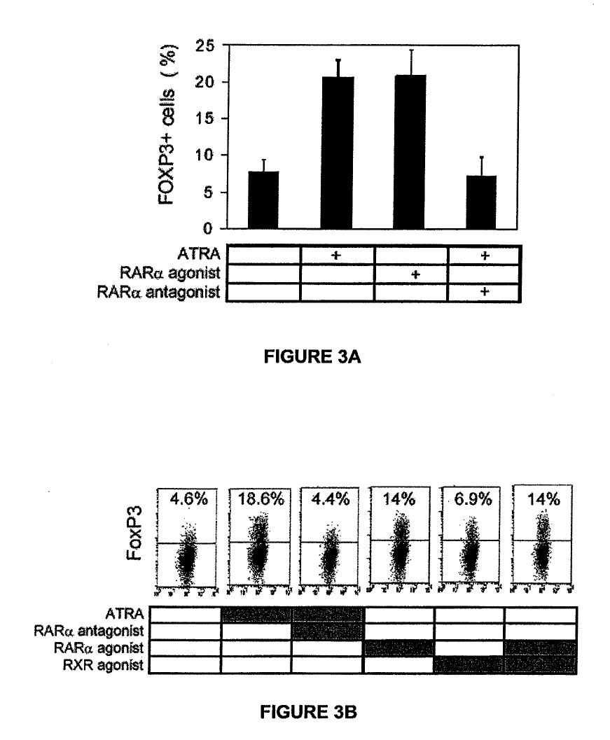

Retinoids induce FoxP3 expression in T cells through RARα. As RARα can be induced in T cells in response to ATRA, the function of this receptor in induction of FoxP3 was examined using an RARα antagonist, Ro41-5253. The RARα antagonist, Ro41-5253 completely suppressed the FoxP3⁺ cell induction effect of ATRA. An RARα, but not the RXR, agonist (methoprene acid), was able to induce FoxP3. FoxP3 protein expression in CD4 T cells, determined by intracellular staining by PE-labeled anti-FoxP3 antibody, is shown as FACS dot plots (FIG. 3A) and graphs (FIG. 3B). Neonatal human cord blood CD4⁺CD25⁻ T cells were cultured in a T cell activation condition with IL-2 (25 U/ml ) and PHA (5 μg/ml) in the presence and absence of indicated agonists or antagonists for 6 days. The concentrations used to obtain the data were 2 nM (ATRA), 1 μM (Ro41-5253), 5 nM (AM-580), and 10 μM (methoprene acid). Data from four independent experiments were combined (FIG. 3B).

The RARα antagonist completely suppressed the induction of FoxP3 by ATRA (FIGS. 3A and B), suggesting that RARα is a dominant receptor that mediates the effect of ATRA. Although RARs are major receptors for ATRA, it is possible that metabolites of ATRA would bind RXRs to induce FoxP3.

Therefore, a RARα-specific agonist, AM-580, was also employed to induce FoxP3. In a manner similar to ATRA, AM-580 was able to induce FoxP3 (FIGS. 3A and B). The optimal concentration of AM580 for induction of FoxP3 was 5 nM (not shown). A pan-RXR agonist, methoprene acid, had only marginal effects on induction of FoxP3 even at a very high concentration (20 μM, FIG. 3A). The two agonists when combined did not induce FoxP3 at higher than the levels induced by the RARα agonist alone. Taken together these data support the role of RARα in ATRA-induced FoxP3 expression.

### EXAMPLE 4

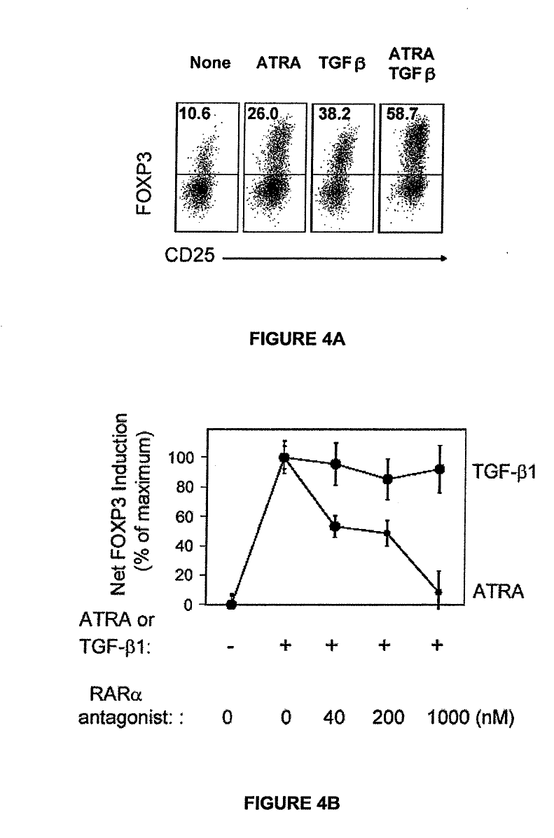

Retinoids enhance the effect of TGF-β in induction of FoxP3. TGF-β is a potent inducer of FoxP3 (Chen et al., 2003, J Exp Med). Therefore, the interaction between ATRA and TGF-β in their effects on FoxP3 expression was examined. FIGS. 4A and 4B show that ATRA and TGF-β cooperate in induction of₁ FoxP3⁺ T cells (FIG. 4A), and FoxP3 induction by TGF-β1 (2 ng/ml) is not dependent on RARα (FIG. 4B). Neonatal human cord blood CD4⁺CD25⁻ T cells were cultured in a T cell activation condition with IL-2 (100 U/ml) and PHA (5 μg/ml) in the presence and absence of indicated agonists or antagonists for 6 days. Ro41-5253 is a RARα antagonist. A representative data set out of four independent experiments is shown. ATRA was used at 2 nM.

Both ATRA and TGF-β had clear effects on induction of FoxP3⁺ T cells, and ATRA, when added to the culture with TGF-β enhanced the effect of TGF-β (FIG. 4A). The RARα antagonist, Ro41-5253, blocked only ATRA-induced but not TGF-β-induced FoxP3 induction (FIG. 4B).

### EXAMPLE 5

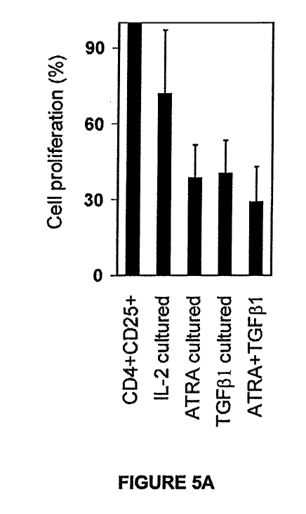

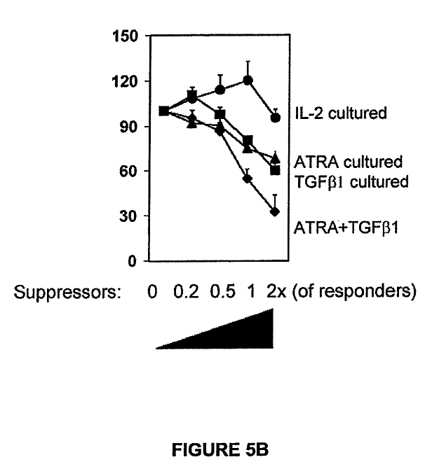

ATRA-treated T cells are suppressor cells with unique effector molecules. Generally, regulatory T cells are hypo-proliferative and can suppress the proliferation of target T cells (CD4⁺CD25⁻ T cells) in co-culture. It was therefore determined if the ATRA-induced FoxP3⁺ cells had these features. FIGS. 5A and 5B show that ATRA-treated T cells are hypo-proliferative (A) and suppressive in function (B). Freshly isolated CD4⁺CD25⁻ T cells were cultured with ATRA (2 nM), TGF-p31 (2 ng/ml) or ATRA+TGF-β1 in a T cell activation condition (IL-2 +PHA) for 6-7 days. Then, the T cells were co-cultured with CD4⁺CD25⁻ cells for 5 additional days. A 3H-thymidine incorporation assay was performed to assess the proliferation potentials of the cultures.

Similar to TGF-β1-treated cells, ATRA-treated T cells had a low proliferation potential compared to fresh CD4⁺CD25⁻ cells and cultured control cells. ATRA+TGF-β1-treated cells even had lower proliferative potentials. ATRA-treated T cells had suppressive activity on CD4⁺CD25⁻ T cells at levels similar to that of TGF-β1-treated cells. TGF-β1+ ATRA-treated cells were most suppressive among the three (FIGS. 5A-5B). Overall, the suppressive activities of the cells were closely correlated with their FoxP3⁺ cell frequencies or the FoxP3 induction activities of the agonists.

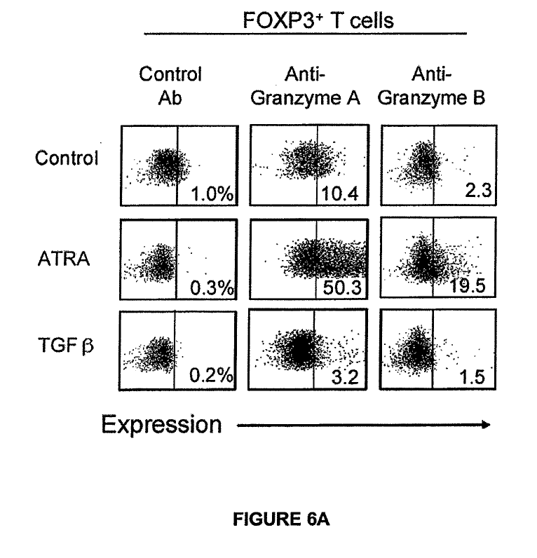

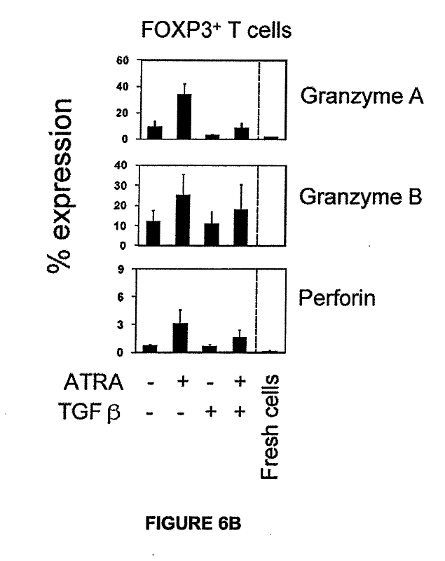

Regulatory T cells may use a number of different effector mechanisms to suppress target cells. Granzymes are a class of such effector molecules implicated in regulatory function. FIGS. 6A and 6B show that ATRA-treated FoxP3⁺ T cells uniquely express granzymes as potential effector molecules. FoxP3⁺ T cells, generated from cord blood CD4⁺CD25⁻ T cells with ATRA, were examined for their expression of granzyme A, granzyme B and perforin. Also included were FoxP3⁺T cells, generated with IL-2 (control) or IL-2 (25 U/ml) and TGF-β (2 ng/ml). Expression of granzyme A and B is shown in panel A. Three independent experiments were combined and averages and SEM are shown (panel B).

Interestingly, many ATRA-induced FoxP3⁺ cells expressed granzyme A, while freshly isolated FoxP3⁺ cells or FoxP3⁺ cells induced with TGF-β1 alone did not express granzyme A at significant levels (FIG. 6A). Consistently more granzyme B or perforin-expressing cells were found among the vitamin A induced FoxP3⁺ cells than TGF-β induced FoxP3⁺ cells, suggesting vitamin A (ATRA) induces a unique class of regulatory cells in effector molecules (FIG. 6B).

### EXAMPLE 6

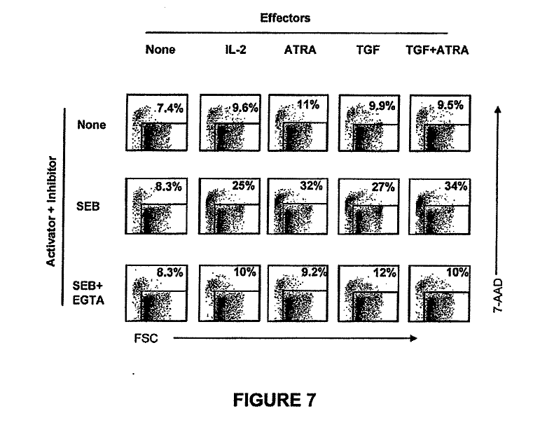

Retinoids induce FoxP3⁺ cells with homing receptors for mucosal tissues. The migration ability of T cells is regulated by the trafficking receptors expressed by the T cells and determines the tissue sites of T cell effector function. Therefore the homing receptor phenotype of retinoid-induced FoxP3⁺ cells was examined and the results shown in FIG. 7. FoxP3⁺T cells, generated from cord blood CD4⁺CD25⁻ T cells with IL-2 (25 U/ml), ATRA (2 nM), TGFβ1 (2 ng/ml) or ATRA+TGFβ1, were examined for cell killing activity. PHA-activated B cells were used as target cells at a target:effector ratio of 1:10. EGTA was added to suppress perforin-dependent cell killing activity.

Retinoid-induced FoxP3⁺ cells expressed a number of trafficking receptors: homeostatic secondary lymphoid (CCR7 and L-selectin), antigen-induced inflammatory (CCR4 and CXCR3), and mucosal tissue homing receptors (CCR9 and integrin β7chain). Only the mucosal tissue homing receptors (CCR9 and integrin β7chain) were uniquely expressed by the retinoid-induced FoxP3⁺ cells compared with control (IL-2-induced) FoxP3⁺ cells.

### EXAMPLE 7

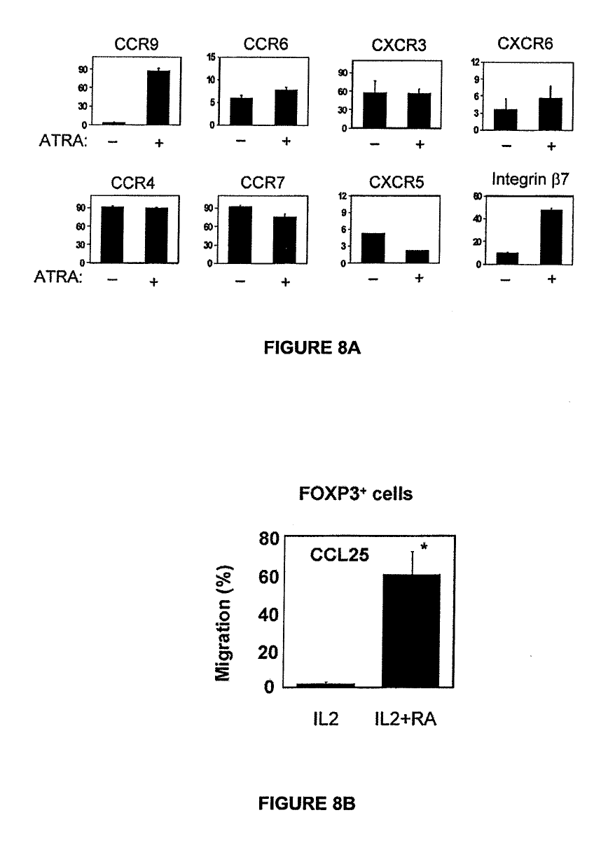

Retinoid-induced FoxP3⁺ cells have cell killing activities. To determine the cell killing activity, FoxP3⁺ T cells, generated from cord blood CD4⁺CD25⁻ T cells with IL-2, ATRA, TGF β1 or ATRA TGF β1, were examined for cell killing activity. Neonatal human cord blood CD4⁺CD25⁻ T cells were cultured in a T cell activation condition (IL-2 and PHA; 100 U/ml and 5 mg/ml respectively) in the presence and absence of ATRA (2 nM) for 6 days. Cells were stained with monoclonal antibodies to chemokine receptors for flow cytometry analysis (FIG. 8A). Chemotaxis assay to the mucosal tissue chemokine CCL25 (5 mg/ml) was performed using a Transwell migration assay (FIG. 8B). Three independent experiments were combined and averages and SEM are shown.

The retinoid-induced FoxP3⁺ cells had efficient cell killing activity towards B cells as shown in FIGS. 8A and 8B. This cell killing activity was inhibited by EGTA (an inhibitor of perforin pathway), suggesting that the cells kill target cells in a perforin-dependent manner.

### EXAMPLE 8

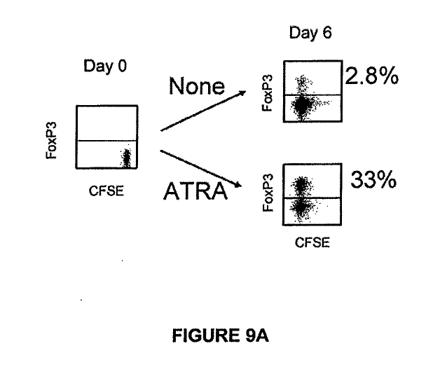

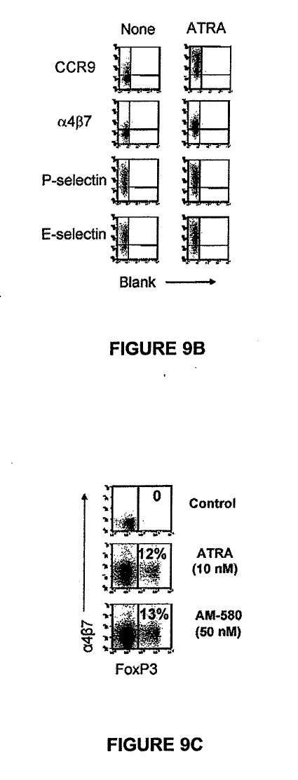

The FoxP3 induction effect of retinoids is conserved in both humans and mice. It was determined if ATRA could induce FoxP3 in mouse T cells. DO11.10⁺ (Rag2−/−) mice do not have FoxP3⁺ T cells, because the antigen (ovalbumin) specific for DO11.10 T cells is not expressed in the mice (Walker et al., 2003, J Exp Med). These CD4⁺ CD25⁻ T cells were cultured in a T cell activation condition (irradiated splenocytes with 2 mg/ml of OVA ₃₂₃₋₃₃₉ antigen peptide and 100 U/ml of IL-2) in the presence or absence of ATRA. Similar to human T cells, ATRA induced FoxP3 expression in T cells (FIG. 9A). Mouse FoxP3⁺ T cells, primed in retinoic acid, expressed the mucosal tissue homing receptors CCR9 and α4β7 as unique trafficking receptors (FIG. 9B). Other retinoids such as AM-580 (a RARα agonist) and methoprene acid (a RXR agonist) were also tested. AM-580 was a good inducer of FoxP3 at low concentrations (FIG. 9C). Methoprene acid was able to induce FOXP3 only at very high concentrations (optimal dose=˜100 μM). FIGS. 9A-9C show a representative data set out of four independent experiments is shown.

### EXAMPLE 9

Retinoid-induced FoxP3⁺ cells selectively migrate to several mucosal tissues. The in vivo homing behavior of the retinoid-induced FoxP3⁺ T cells was determined. For this, retinoid-induced FoxP3⁺ cells were prepared from CD4⁺CD25⁻ T cells as described in Example 8, and injected intravenously into host mice. Mice were sacrificed 20 hours later, and migration of injected FoxP3⁺ cells and FoxP3⁻ T cells to 10 different tissue sites was determined. Control T cells were labeled with the red fluorescent dye tetramethylrhodamine-5-(and-6)-isothiocyanate (TRITC), while test T cells were labeled with the green dye 5-(and-6)-carboxyfluorescein diacetate succinimidyl ester (CFSE). The two T cell subsets (10⁷ cells per mouse) were co-injected into BALB/c mice via a tail vein. The lamina propria lymphocytes (LPLs) were isolated after removing the epithelial cells with 5 mM EDTA (5 times), digestion (3 times, 45 min each) with 300 U/ml collagenase (type 3, Worthington, Lakewood, N.J.) and 100 μg/ml DNase I (Worthington), filtration through a nylon mesh, and centrifuge in a 40/75% Percoll-gradient. The cells were stained with antibodies to CD4 and FoxP3. Normalized homing indexes were calculated by a formula (HI=a÷b), where a=[test cells migrated into the tissue site]=[control cells migrated into the tissue site], and b=[test cells in input]÷[control cells in input]. LP (SI) and LP (LI) are lamina propria of small and large intestine respectively. “P. cavity” is peritoneal cavity, and “Peyer's p.” is Peyer's patches. Three independent experiments were combined and averages and SEM are shown.

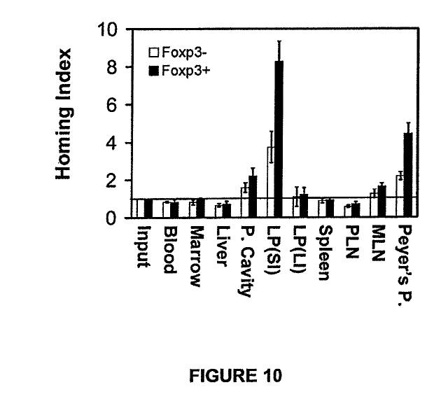

As shown in FIG. 10, the retinoid-induced FoxP3⁺T cells preferentially migrate to the small intestinal lamina propria and Peyer's patches among the tissue sites examined. Migration to peritoneal cavity and lymph nodes (MLN) was also slightly elevated for retinoid-induced compared to control FoxP3⁺ T cells. Interestingly, FoxP3⁺ CD4 T cells were more efficient in migration to the mucosal tissue sites than FoxP3⁻ cells.

### EXAMPLE 10

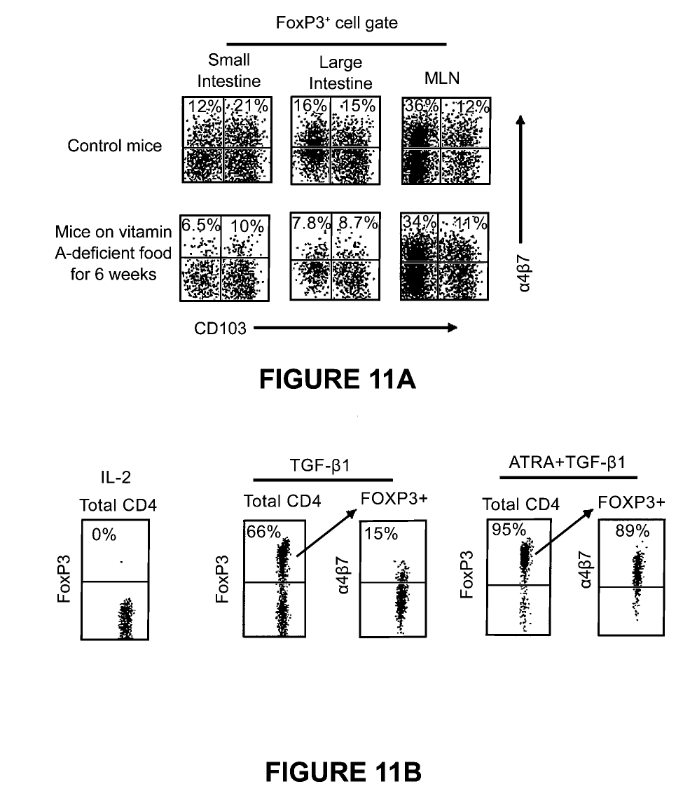

Regulation of mucosal FoxP3⁺ cell numbers by vitamin A in vivo and in vitro. The in vivo effect of vitamin A on numbers of intestinal FoxP3⁺ T cells was determined. Adult mice were fed with food with (control mice) or without vitamin A for 6 weeks, and then the numbers of FoxP3⁺ T cells in small and large intestine, and mesenteric lymph nodes were determined by flow analysis (FIG. 11A). The mice fed with vitamin A-deficient food had significantly smaller numbers of α4β7⁺ mucosal tissue homing FoxP3⁺ T cells in the large and small intestines compared to the mice fed with control food having vitamin A. However, there was no reduction in lymph nodes (MLN), demonstrating a tissue specificity. These data demonstrate that the induction of FoxP3⁺ cells by vitamin A and retinoids takes place in vivo.

Also provided is a method with which almost all of naïve T cells can be converted into mucosal tissue homing FoxP3⁺ T cells by utilizing ATRA and cytokines. Mouse naïve T cells were cultured with ATRA (1-100 nM), IL-2 (10-1000 U/ml) and TGF-β1 (1-100 ng/ml), together with TCR activators (concanavalin A or PHA at 1-10 μg/ml) for 6-7 days. FIG. 11B shows that ˜95% of T cells after culture were FoxP3⁺ regulatory T cells expressing the mucosal tissue homing receptor αβ7. For comparison, the control culture generated fewer FoxP3⁺ T cells and these cells did not express α4β7. This method reproducibly generates 92-99% pure mucosal tissue homing FoxP3⁺ T cells.

### EXAMPLE 11

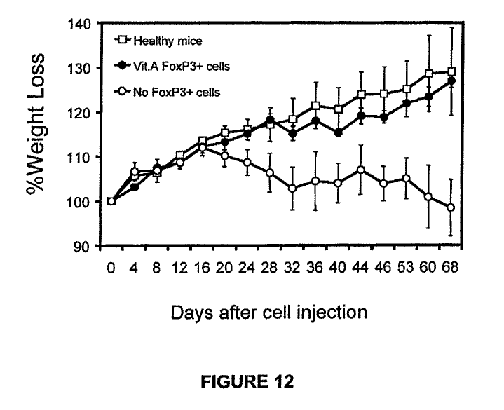

Regulation of inflammation by retinoid-induced FoxP3⁺ T cells in vivo. The immune regulatory function of retinoid-induced FoxP3⁺ T cells was determined in vivo. FIG. 12 shows that retinoid-induced FoxP3⁺ regulatory T cells can suppress the induction of inflammation in mice. A mouse model of inflammation (SCID mouse) was utilized, which is commonly used as a general animal model of inflammatory immune responses. These mice lose weight and eventually die when they are injected with inflammatory T cells (CD4⁺CD62L⁺CD45RB⁺/mouse). The major cause of the weight loss is the inflammation in the intestine. SCID mice were injected with inflammatory T cells (0.5×10⁶ CD4⁺CD62L⁺CD45RB⁺/mouse) to induce intestinal inflammation. At the same time, some mice were injected with retinoid-induced FoxP3⁺ regulatory T cells (0.5×10⁶/mouse). Control SCID mice are the mice that were not treated. It should be noted that the weight loss due to the inflammation was effectively suppressed by the retinoid-induced FoxP3⁺ regulatory T cells (vitamin A FoxP3⁺ cells). Each group contained 3-5 mice. The data in FIG. 12 show that the weight loss in these mice due to the inflammation was effectively suppressed by the retinoid-induced FoxP3⁺ regulatory T cells. The effective dose was 0.5×10⁶ cells/mouse injected intravenously. This dose may be even lower and the same efficacy obtained.

### EXAMPLE 12

Regulation of anti-inflammatory T cell numbers in vivo by controlling vitamin A consumption and retinoic acid concentration. BALB/c mice were fed with rodent diets containing a normal level of retinol acetate (2500 U/kg; VAN), excessive level (25,000 U/kg; VAS) or no retinol acetate (0 U/kg; VAD) for 12 weeks following birth. At 12 weeks, naïve CD4⁺T cells from D011.10 rag2−/− mice were transferred into the VAN, VAS, and VAD mice and the mice were immunized intraperitoneally with ovalbumin (500 μg/mouse) in incomplete adjuvant. All-trans retinoic acid (1 mg/ kg) was injected intraperitoneally on day 0, 2, 4, and 6. The frequencies of mucosal FoxP3⁺T cells (% of CD4⁺ T cells) were determined in various organs 7 days post immunization. This experiment provided a method to control the numbers of suppressor T cells (i.e. anti-inflammatory T cells) in vivo by controlling the consumption of vitamin A and concentration of retinoic acid. Abbreviations of organ names: MLN (mesenteric lymph node), Per. Cavity (peritoneal cavity), PLN (peripheral lymph node), PP (Peyer's patches), and Col. Patches (colonic patches).

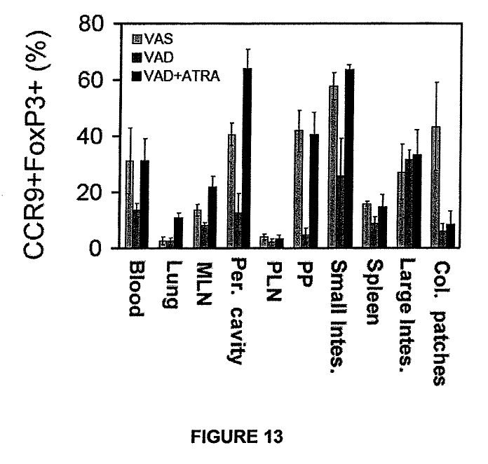

FIG. 13 is an example that the numbers of anti-inflammatory T cells can be increased or decreased in the body by controlling the consumption of vitamin A. More specifically, by lowering the vitamin A/retinoic acid concentrations, the numbers of anti-inflammatory T cells in mucosal tissues can be decreased. Lowering the vitamin A/retinoic acid concentrations in the body can be achieved through special diet with reduced vitamin A or through administering retinoid receptor antagonists. By increasing the concentrations, the numbers of the anti-inflammatory T cells can be increased. Increasing the retinoid concentrations in the body can be achieved through use of fortified vitamin A supplements or through administering retinoid receptor agonists. It has been shown that these anti-inflammatory T cells induced by retinoic acid are highly effective in suppression of tissue inflammation (FIG. 12).

It should be understood, of course, that the foregoing relates to exemplary embodiments of the invention and that modifications may be made without departing from the spirit and scope of the invention as set forth in the following claims.

1. A method for generating mucosal tissue homing immunosuppressive T-cells comprising treating naive T-cells with a retinoid or a retinoid receptor agonist.

2. The method of claim 1 wherein the retinoid is retinol palmitate, retinyl palmitate, retinol acetate, beta-carotene or combinations thereof.

3. The method of claim 1 wherein the retinoid is all-trans-, 9-cis-, 11-cis retinoic acid or combinations thereof.

4. The method of claim 1 wherein the retinoid receptor agonist is AM-580, BMS641, BMS961, CD666, TTNPB, ATRA, Ro25-7386, methoprene acid or combinations thereof.

5. The method of claim 1 wherein the retinoid or retinoid receptor agonist has a concentration of from about 100 pM to about 1 mM.

6. The method of claim 1 wherein the retinoid or retinoid receptor agonist has a concentration of from about 100 pM to about 100 nM.

7. The method of claim 1 wherein the mucosal tissue is alimentary tract, respiratory tract, lung, stomach, intestines or colon.

8. The method of claim 1 wherein the naïve T-cells are obtained from cord blood, peripheral blood, secondary lymphoid tissue or combinations thereof.

9. The method of claim 1 wherein the naïve T-cells are treated ex vivo.

10. The method of claim 1 wherein the naïve T-cells are treated in vivo.

11. The method of claim 1 further comprising the reduction or prevention of inflammation in a mucosal tissue by the generated immunosuppressive T-cells.
